(19) United States
(12) Patent Application Publication
(10) Pub. No.: US 2016/0100973 A1
(43) Pub. Date: Apr. 14, 2016

(54) Flexible Wearable Foot Sling

(71) Applicant: Miriam S. Nelson, Asheville, NC (US)

(72) Inventor: Miriam S. Nelson, Asheville, NC (US)

(21) Appl. No.: 14/881,812

(22) Filed: Oct. 13, 2015

Related U.S. Application Data
(60) Provisional application No. 62/063,139, filed on Oct. 13, 2014.

(51) Int. Cl. A61F 5/01

(52) U.S. Cl. CPC A61F 5/0127

(57) ABSTRACT

A wearable foot sling includes a front end, the front end being comprised of webbing that is sized and configured to fit over the interdigit spaces of the metatarsals of a user; a back end, the back end being sized and configured to receive and secure therein the user's posterior heel; the front end and the back end being joined together; and wherein, once positioned on a user's foot, the sling functions to restrain the longitudinal lengthening of the soft tissues between the user's metatarsals and heel.

### RELATED APPLICATION

This application claims priority to and incorporates entirely by reference U.S. Provisional Patent Application Ser. No. 62/063,139 filed on Oct. 13, 2014.

### FIELD OF THE INVENTION

This invention relates to medical devices and, more particularly medical devices that support and assist individuals with musculoskeletal foot pain.

### BACKGROUND OF THE INVENTION

Human feet and ankles bear the brunt of every step we take. Women in particular wear uncomfortable, jarring yet fashionable shoes that can do damage to the structure of the foot over time. Athletes are also at risk of heightened foot problems from traumatic injury, joint, tendon, or ligament problems.

Both arthritis and foot pain are major public health problems. Approximately 24% of adults have foot ailments, and the prevalence increases with age. Surprisingly, this topic of musculoskeletal foot pain has received little attention in the rheumatology community.

Some of the most common foot problems experienced by individuals include, but are not limited to, foot and ankle trauma/fractures, ligament sprains/tendonitis, Achilles tendon problems, heel pain/plantar fasciitis, bunions, hammertoes or claw toes, ankle or foot arthritis and flat feet deformities, among other problems.

Despite the major focus of structure and alignment in arthritis, remarkably little work has focused on the foot and nonsurgical foot interventions that might affect lower extremity joint alignment, structure and pain in rheumatic diseases. Emerging research suggests that there may be a significant role for foot orthotics and footwear in the treatment of rheumatoid arthritis, osteoarthritis of the hip, knee and foot and other commonly experienced conditions associated with musculoskeletal foot pain.

Because of these and other problems in the art, described herein, among other things, is a wearable foot sling for the relief of musculoskeletal foot pain comprised of materials possessing elastomeric properties that mimic the anatomical structures of the foot. In certain embodiments, the wearable foot sling described herein mimics the anatomical structures of the foot that may support the medial longitudinal arch and enhance the windlass mechanism during the late stance and push off phases of gait.

### SUMMARY OF THE INVENTION

In accordance with one form of this invention, there is provided a wearable foot sling, the foot sling including a front end, the front end being comprised of webbing that is sized and configured to fit over the interdigit spaces of the metatarsals of a user; a back end, the back end being sized and configured to receive and secure therein the user's posterior heel; the front end and the back end being joined together; and wherein, once positioned on a user's foot, the sling functions to restrain the longitudinal lengthening of the soft tissues between the user's metatarsals and heel.

### BRIEF DESCRIPTION OF THE DRAWINGS

The invention may be better understood in reference to the following description taken in conjunction with the accompanying drawings in which:

FIG. 1 shows a perspective view of an embodiment of the flexible wearable foot sling;

FIG. 2 shows a perspective view of embodiment of the flexible wearable foot sling;

FIG. 3 shows a perspective view of an embodiment of the flexible wearable foot sling; and

FIG. 4 shows a perspective view of an embodiment of the flexible wearable foot sling.

Like reference numerals refer to like parts throughout the several views of the drawings.

### DETAILED DESCRIPTION OF THE PREFERRED EMBODIMENTS

In general, the wearable foot sling (101) disclosed herein is designed to address relief of musculoskeletal foot pain and is designed for comfort and compatibility with most shoes worn, including low profile dress and sport shoes.

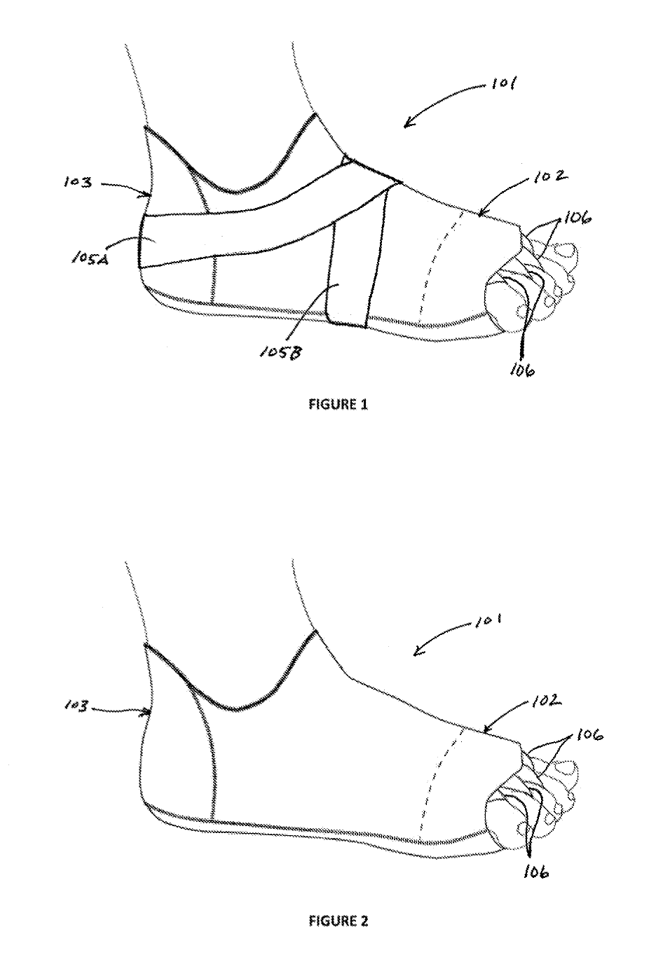

In certain embodiments, the wearable foot sling (101) will be comprised of a conforming material with elastomeric properties known to those of ordinary skill in the art. Contemplated materials include, but are not necessarily limited to, neoprene, polychloroprenes and other known synthetic rubbers with elastomeric properties. In one embodiment, as depicted in FIGS. 1-2 the wearable foot sling (101) is comprised of a tight-fitting sling (101), the sling (101) having a front end (102) which is comprised of webbing (106) that fits over the interdigit spaces of toes 1-5 and a back end (103) which is secured over the posterior heel. Once the sling (101) is positioned in place on a user's foot, the sling (101) functions to restrain the longitudinal lengthening of soft tissues between a user's toes and heel, effectively shortening the fibers of the plantar fascia.

In certain alternative embodiments, the sling (101) will include bands of elastomeric properties aligned in parallel with the metatarsals that will augment the shortening effect applied by the sling (101). In general, any material known to those of ordinary skill in the art with elastomeric properties is contemplated as a possible band aligned in parallel with the metatarsals.

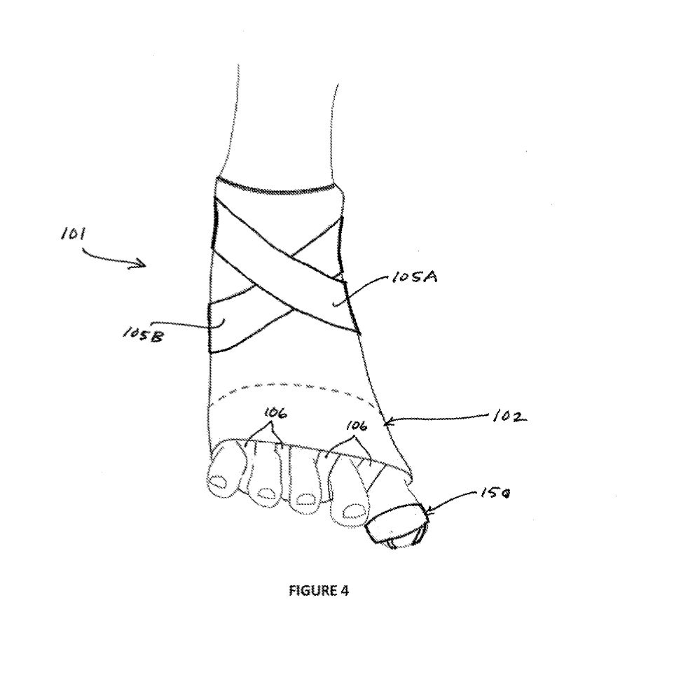

In other embodiments of the sling (101), there will be a pair of diagonally oriented supports 105A, 105B) that cross under the plantar midfoot to further support the medial longitudinal arch. In certain embodiments, as demonstrated in FIGS. 1 and 4, the diagonally oriented supports (105A, 105B) will also cross diagonally on the top of the foot and sling (101), although this orientation is not determinative. As depicted in FIGS. 1 and 4, the medial longitudinal diagonally oriented supports (105A, 105B) will likely be secured over the dorsum of the foot by fastening means known to those of ordinary skill in the art including, but not limited to, hook and loop fastener, clips, hooks, ties, fasteners, knots, and loops. In one embodiment, the diagonally oriented supports (105A, 105B) are made from an elastomers material.

In certain additional embodiments, an additional element of the sling (101) is a smaller sling system around the first phalanx of the great toe, the phalanx sling (150). As demonstrated in FIG. 4, the phalanx sling (150) is comprised of a single support that attaches to the bottom of the sling (101) at the base of the great toe, wraps around the front of the great toe and then crosses back over itself to attach to the bottom of the sling (101) at the base of the great toe on the opposite side. Accordingly, the phalanx sling (150) wraps around the front of the toe and forms a cross and attaches to the bottom of the sling near the great toe. In general, the function of the phalanx sling (150) is to improve plantar flexion during loading. It should be understood that this phalanx sling (150) may be optional, and could be reversed (i.e., the phalanx sling may be wrapped around the great toe in the opposite manner than demonstrated in FIG. 4) to enhance dorsiflexion.

In sum, the orientation of the sling (101) provided herein creates a multi-point harness which shortens and restricts the motion of the plantar structures of the foot in a triplanar manner, thereby decreasing abnormal tension in muscle, fascia, and neural pathways. As such, this device is similar in purpose to both the molded and non-molded foot orthoses of the prior art which are utilized for decreasing foot pain.

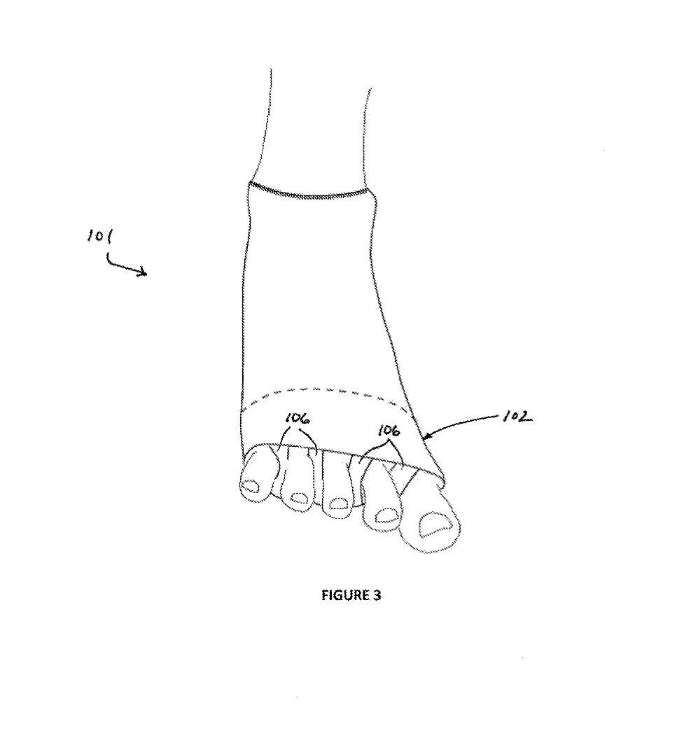

However, the sling (101) described herein has numerous advantages over these designs of the prior art. First, as demonstrated in FIGS. 1-4, the sling (101) has a lower profile, fitting snugly with the foot. This lowered profile allows compatibility between the sling (101) and most shoe designs. Further, unlike the designs of the prior art, because of its low profile a user can use the sling generally without removing the original insoles of the shoes. Second, the sling offers the possibility for increased customer compliance and more frequent use than the currently utilized methodologies due to decreased sense of difficulty with use (compared to prior art technologies) and increased sense of comfort. Third, due to its simple design, the sling (101) is more reasonably priced than the custom foot orthoses of the prior art. In addition, the currently utilized over-the-counter and custom orthoses frequently have to be accommodated by either the purchase of a separate pair of shoes to account for increased depth and width of the orthoses materials, also adding to the cost. As noted previously, this is not necessary with the disclosed sling (101). Finally, another issue with current foot orthoses utilized in the art is the incompatibility of a person's foot function and the density and contours of the materials comprising the orthoses. Pain discomfort and blisters are frequent results of such issues with errors in fit and material density. Non-compliance by patients due to discomfort and cost caused by the orthoses of the prior art is a frequently cited drawback in some studies on foot pain and orthoses.

In certain embodiments, the sling (101) disclosed herein will be utilized as follows. In a first step, the front end (102) of the sling, which is comprised of webbing (106) that fits over the interdigit spaces of toes 1-5, is placed in the interdigit spaces of a user's toes. Generally this first step is performed while the user is in a non-weight bearing position (e.g., sitting). In a second step, the back end (103) of the sling (101) is secured over the user's posterior heel, bringing the sling into a snug, taut and comfortable position on the bottom of a user's foot. In a third step, the user secures the diagonally oriented supports (105A, 105B) (proximal/lateral to distal/medial and distal/lateral to proximal/medial midfoot) to create a hammock for the navicular. In a fourth step, in the embodiments of the sling (101) with a phalanx sling (150), the phalanx sling (150) is positioned into place around the great toe. In these embodiments, this phalanx sling assists the proximal phalanx of the first toe in plantar flexion, but does not necessarily restrict overall mobility. As noted previously, this phalanx sling (150) may be reversed to the dorsum or removed based on the comfort to the customer.

While the invention has been disclosed in connection with certain preferred embodiments, the elements, connections, and dimensions of the preferred embodiments should not be understood as limitations on all embodiments. Modifications and variations of the described embodiments may be made without departing from the spirit and scope of the invention, and other embodiments should be understood to be encompassed in the present disclosure as would be understood by those of ordinary skill in the art.

What is claimed is:

1. A wearable foot sling, said foot sling comprising:
a front end, said front end being comprised of webbing that is sized and configured to fit over the interdigit spaces of the metatarsals of a user;
a backend, said back end being sized and configured to receive and secure therein the user's posterior heel;
said front end and said back end being joined together; and
wherein, once positioned on a users foot, the sling functions to restrain the longitudinal lengthening of the soft tissues between the user's metatarsals and heel.

2. The wearable foot sling of claim 1 wherein said front end is made from an elastomeric material.

3. The wearable foot sling of claim 1 wherein said back end is made from an elastomerrc material.

4. The wearable foot sling of claim 1 wherein said wearable foot sling further comprises a plurality of bands, and said plurality of bands being in parallel alignment with the metatarsals.

5. The wearable foot sling of claim 1, said wearable foot sling further comprising a pair of diagonally oriented supports, the supports crossing under the plantar midfoot and being secured over the dorsum of the foot by a fastening means.

6. The wearable foot sling of claim 5, wherein the fastening means comprises a hook and loop fastener.

7. The wearable foot sling of claim 1 further comprising a phalanx sling, and said phalanx sling being sized to receive the great toe of the user's foot for improving plantar flexion during loading.

8. The wearable foot sling of claim 7 wherein said phalanx sling is made from an elastomeric material.

9. The wearable foot sling of claim 1, wherein the wearable foot sling has a low profile.
